(19) United States
(12) Patent Application Publication
(10) Pub. No.: US 2022/0105293 A1
(43) Pub. Date: Apr. 7, 2022

(54) RESPIRATOR FACE MASK POSITIONING AND RETENTION DEVICE

(71) Applicant: Richard Garcia, Visalia, CA (US)

(72) Inventor: Richard Garcia, Visalia, CA (US)

(21) Appl. No.: 17/495,743

(22) Filed: Oct. 6, 2021

Related U.S. Application Data
(60) Provisional application No. 63/088,008, filed on Oct. 6, 2020.

(51) Int. Cl. A61M 16/06

(52) U.S. Cl. CPC A61M 16/0605; A61M 2210/0606; A61M 2210/0618; A61M 16/0688

(57) ABSTRACT

The present invention is a device that will properly position a respiratory face mask on a user's face and reduces the likelihood that it will slip downward off the subject's nose exposing the wearer's nostrils and negating the mask's intended purpose of stopping the transmission of airborne diseases.

### FIELD OF THE INVENTION

The present invention relates generally a device for alleviating the frustrating problem of mask slippage. More particularly, the present invention provides a device operable to prevent mask shifting/slippage.

### BACKGROUND OF THE INVENTION

Keeping a face mask properly positioned on a user's face can be problematic. For example gravitational forces and facial movements, particularly while talking, can cause the mask to move and migrate downward. This problem is particularly concerning since masks are the frontline defense to stop the spread of diseases like Covid 19. The downward migration of the mask will expose the user's nostrils and negate the mask's intended purpose to stop the transmission of airborne viruses and other diseases. The slipping mask will invariably result in the user attempting to readjust the mask using their fingers, potentially transferring pathogens from the hands of the user to their mouth or eyes. This invention will reduce the need to partake in such unsanitary practice.

Some mask wearers have attempted to prevent mask slippage by wearing their eye glasses over the top edge of their mask, others have tried to address the problem by using tape to fasten the edges of the mask to their face. Keeping a face mask properly in place on a young active person can be a challenge, especially in a classroom situation. Thus, a device operable to prevent mask shifting/slippage is needed.

### SUMMARY OF THE INVENTION

The present invention is a device that will properly position a repertory face mask on a user's face and reduces the likelihood that it will slip downward off the subject's nose exposing the wearer's nostrils and negating the mask's intended purpose of stopping the transmission of airborne diseases. The face mask is positioned and held in place by a grasping material (e.g., hook-and-loop fastening material) operable to attached to a mask worn over the face, a latching pad which is fastened to a non-toxic tape with adhesive in conjunction, and a spacer/buffer/release pad that serves three functions. The pad may elevate the latching pad above the surrounding tape to create a gap exposing the sharp edges of the latching pad improving mask engagement. The pad also serves as a buffer to insulate the skin from the adhesive used to join the latching pad to the tape and is designed to provide a convenient accessible place to grab the device to safely remove it.

The present invention is a novel device comprised of a strip of adhesive material (e.g., a tape) that is attached to a component called a latching pad that has an outward facing grasping material (e.g., hook and loop fastener material like Velcro®, a rough material coarse sand paper, a material having short protruding bristles, or a similar material). The latching pad may be fastened to the adhesive tape in such a way that it would afford good engagement with the face mask. The wearer fastens the strip of tape with the attached latching pad to their nose prior to positioning the respirator mask on their face. The affixed tape would position the attached latching pad on the lower portion of the bridge of the nose. The latching pad would engagement or “grab” the material of the internal surface of the mask keeping it in place and correctly positioned on the mask wear's face. A fabric's drag coefficient would affect performance. A silk mask might be harder to hold on to than a wooly one. The mask's shape, fabric and seam design would impact performance. In some embodiments, the present invention may further include a fastening pad operable to be adhesively attached to the interior surface of a face mask to provide an anchoring point for the latching pad.

The design of the present invention is such that it will afford the wearer a convenient purchase to easily grab the tape and to safely remove it. In some examples, the adhesive material layer may include pull tabs that do not include adhesive and provide a graspable protrusion that the wearer can grasp and pull to remove the device.

In some embodiments, the present invention comprises a face mask positioning and retaining device that secures the mask on the wear's face comprising: a latching pad that is made from the hook component of a hook and loop fastener system, a strip of material (e.g., a tape) that adheres to the user's nose, and a release pad located between the said adhesive tape and the user's skin that comprises a smaller profile than said latching pad, and operable to: function as a spacer to elevate the outer edges of said latching pad above said adhesive tape, function as a chemical buffer between the latching pad/adhesive tape glue join, be mounted in a manner that provides a convenient grip on the apparatus to facilitate the removal of the device. The latching pad may be comprised of material operable to engage with the inner surface of a mask. The latching pad may be fastened to said mounting tape with a fastening system comprised of at least one of the following: stitches, rivets, laser fusion, ultrasonic welding, hot melt wax, rubber cement, epoxy, or adhesive glue. The latching pad may further comprise a field of exposed latching hooks made of fine wire or cast plastic prongs protruding upwards. The latching pad may be fastened to the mounting tape by at least one adhesive. For example, the at least one adhesive may be a pressure sensitive adhesive that is coated on the back side of the said latching pad. The mounting tape is comprised of a non-allergenic adhesive. The fastening system may further comprise a lamination or injection mold joining process. The latching pad may further comprise a base comprised of said latching hooks and operable to provide a surface to latch onto said mounting tape. The latching pad may be placed above the mounting tape in such a manner that said latching pad overhangs said mounting tape creating a gap exposing the shape edges of said latching pad base increasing the gripping power of the device. The release pad may have exposed edges to provide a secure grip on the device to enable to wearer to safely remove the device.

In some embodiments the present invention comprises a respirator face mask positioning and retaining device that secures the mask on the wear's face comprising: a latching pad comprising a fastening system operable to affix the device to a face mask, and a flexible band operable to adheres to the surface of a user's nose. The device may further comprise a padding layer located between said latching pad and said flexible band operable to elevate the latching pad and provide a grip for removing said device. The padding layer may further comprise exposed edges operable to enable the user to safely and easily remove the device by providing a secure grip on the device. The latching pad may be coupled to said flexible band via at least one of the following affixation methods: stitches, rivets, laser fusion, ultrasonic welding, hot melt wax, rubber cement, epoxy, or adhesive glue. The fastening system may comprise a field of exposed latching hooks that protrude outward from the superior side of said latching pad and are operable to latch onto the inner surface of a face mask. The affixation method may comprise a pressure sensitive adhesive that is coated on the inferior side of the said latching pad. The inferior surface of said flexible band may be comprised of a non-allergenic adhesive operable to adhere said flexible band to the surface of user's nose. The latching pad may have an inferior surface comprised of latching hooks operable to latch onto the superior surface of said flexible band.

The above-described objects, advantages and features of the invention, together with the organization and manner of operation thereof, will become apparent from the following detailed description when taken in conjunction with the accompanying drawings, wherein like elements have like numerals throughout the several drawings described herein. Further benefits and other advantages of the present invention will become readily apparent from the detailed description of the preferred embodiments.

### BRIEF DESCRIPTION OF THE DRAWINGS

FIG. 1 provides a top, front, and side view of the present invention noting the three primary components and their orientation to each other, according to an embodiment of the present invention.

FIG. 2 provides a side view illustrating the correlation between the latching pad and the adhesive tape, according to an embodiment of the present invention.

FIG. 3 provides a front view of the device noting the orientation of the release pad under the tape and the gap between the edges of the latching pad and the tape, according to an embodiment of the present invention.

FIG. 4 provides a top and frontal view illustrating the design of the optional release tabs on each end of the device, according to an embodiment of the present invention.

FIG. 5 provides a top view of the product utilizing a tape product that is designed to improve breathing. This design utilizes a triangle shaped latching pad, according to an embodiment of the present invention.

FIG. 6 provides an illustration of the device affixed to the wear's face, according to an embodiment of the present invention.

FIG. 7 provides an illustration showing the device affixed to the wear's face engaging an internal seam inside the mask, according to an embodiment of the present invention.

FIG. 8 provides a rendering of a model with the device mounted on their nose, according to an embodiment of the present invention.

FIG. 9 provides a rendering of a model wearing a face mask over the invention, according to an embodiment of the present invention.

### DETAILED DESCRIPTION

Reference will now be made in detail to certain embodiments of the invention, examples of which are illustrated in the accompanying drawings. While the invention will be described in reference to these embodiments, it will be understood that they are not intended to limit the invention. To the contrary, the invention is intended to cover alternatives, modifications, and equivalents that are included within the spirit and scope of the invention. In the following disclosure, specific details are given to provide a thorough understanding of the invention. However, it will be apparent to one skilled in the art that the present invention may be practiced without all of the specific details provided.

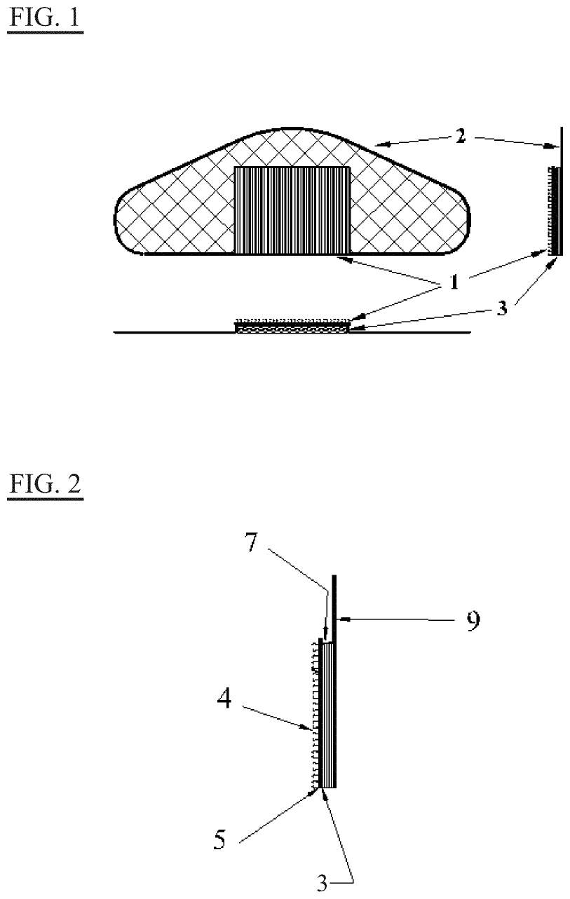

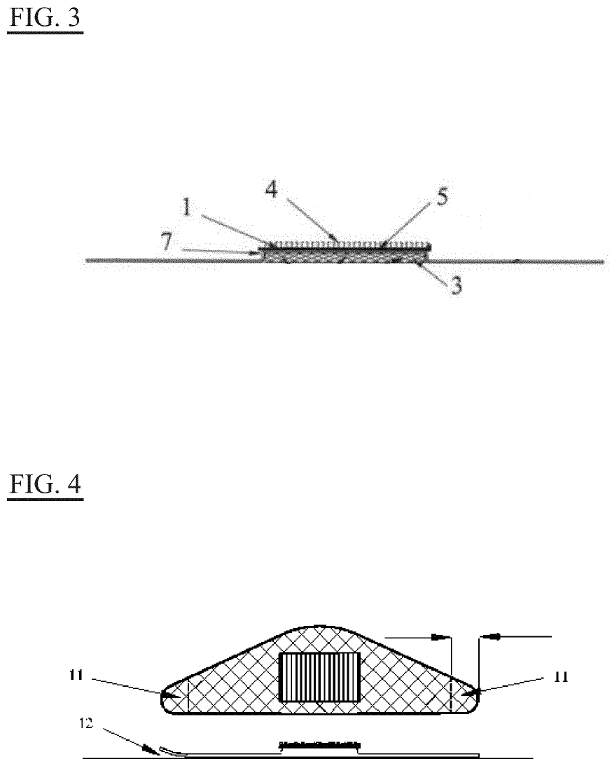

As shown in FIG. 1 and FIG. 2, the present invention comprises a latching pad 1 made from hook-and-loop fastening material or a similar material that may be fastened to a strip of adhesive band 2 (e.g., a tape). The flexible band 2 could be made of paper, cloth, plastic, or fabric similar to bandage products and may further comprise a padding layer 3 between latching pad 1 and flexible band 2, operable to provide a cushioning effect for the user. The flexible band may comprise an adhesive base 9, operable to affix the device to the nose of a user.

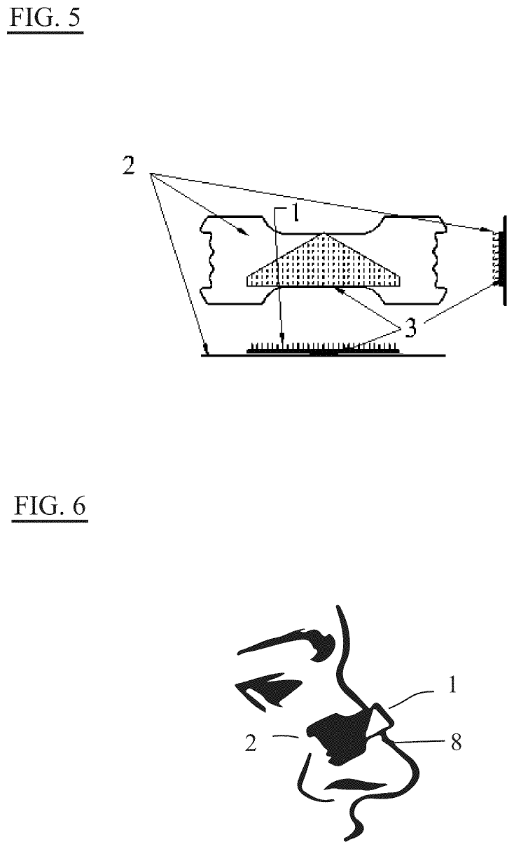

As shown in FIG. 1, the latching pad 1 may have a rectangular shape but may also be configured in any number of shapes and sizes including but not limited to the outline of a rectangle, square, trapezoid (see FIG. 5), triangle, diamond, circle, star or oval. The invention may use the “hook” component 4 of a hook-and-loop fastening system facing outwardly from the nose latching pad 1. The latching pad 1 may be mounted so the open side (i.e., the superior side) is comprised of a field the latching hooks 4 protrude outwardly to allow for engagement with the interior surface of a face mask. In such configuration, the device will increase traction to the inside of the mask, preventing downward shifting of the face mask. The latching pad 1 may be fastened to the outer side of the adhesive band 2 by a fastening system. The fastening system may be one of a number of methods, or a combination thereof, including, but not limited: gluing, sewing, stapling, laser fusing, sonic welding, or lamination. For example, the latching pad 1 may comprise an adhesive 6 operable to affix the latching pad to the flexible band 2. The latching pad may have a base 5 that may act as a foundation for the hooks 4 as well as the fixation system.

As shown in FIG. 4, the flexible band may comprise exposed edges 11 (e.g., pull-tab), that lacks adhesive material and provide an grip for the user to safely and easily remove the device after use. The exposed edges may be folded or bent in a manner such that the device creates a pull tab gap 12, between the elastic band 2 and the surface to which the device has been applied (e.g., the nose of a user).

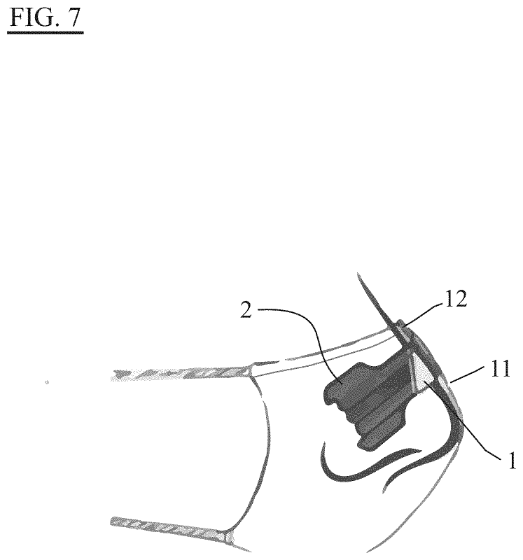

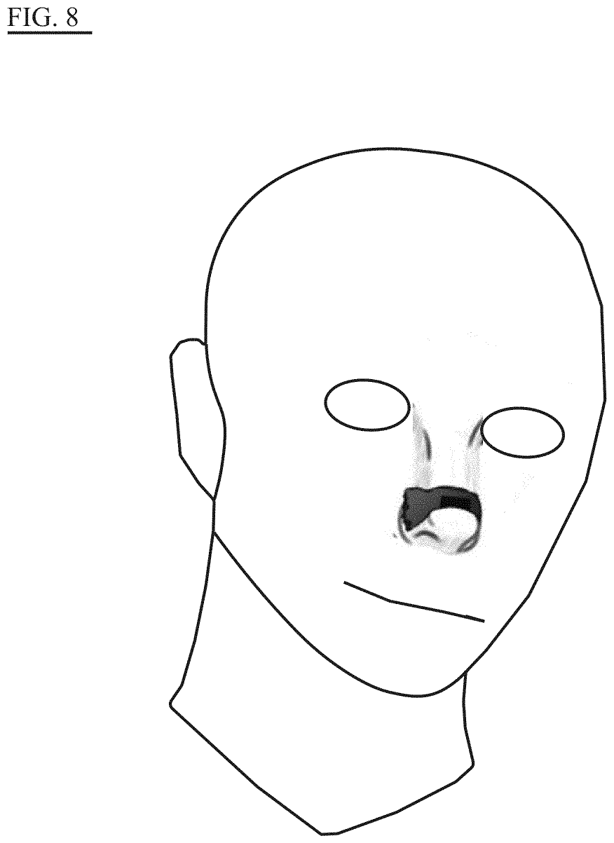

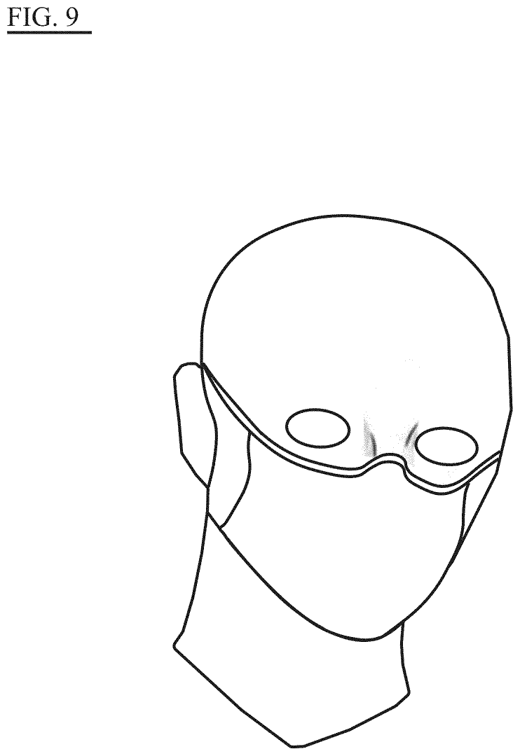

In some implementations, a user may fasten the adhesive band 2 with the attached latching pad 1 to their nose prior to positioning the mask on their face. As shown in FIG. 6, the device may be positioned such that the latching pad 1 protrudes from a portion 8 of the nose. As shown in FIG. 7, the latching pad 1 may be the positioned under the seam 12 of a standard mask.

In some embodiments, as shown in FIG. 6 and FIG. 7, the present invention may comprise a design that effectively grips and effectively fastens the inside of the face mask by protrusions (e.g., hooks) of the latching pad, when the mask is correctly positioned on the face of a user.

In some embodiments, the device may be used either with or without the “loop” component of the system fastened to the mask. A patch of material having protruding loop fasteners may be attached to the interior surface of the mask by adhesive such that the loop fasteners may be engaged with the hook fasteners protruding from the latching pad 1. Some masks, as shown on FIG. 7, may have a strip of foam fabric 11 glued to the inside of the mask that interfaces perfectly with the invention's latching pad 1. When the invention is used without a “loop” type pad mounted inside the mask the product will still afford a significant amount of purchase on the internal surface and seams of the mask helping to keep the mask from slipping.

It is to be understood that variations, modifications, and permutations of embodiments of the present invention, and uses thereof, may be made without departing from the scope of the invention. It is also to be understood that the present invention is not limited by the specific embodiments, descriptions, or illustrations or combinations of either components or steps disclosed herein. The embodiments were chosen and described in order to best explain the principles of the invention and its practical application, to thereby enable others skilled in the art to best utilize the invention and various embodiments with various modifications as are suited to the particular use contemplated. Although reference has been made to the accompanying figures, it is to be appreciated that these figures are exemplary and are not meant to limit the scope of the invention. It is intended that the scope of the invention be defined by the claims appended hereto and their equivalents.

What is claimed is:

1. A respirator face mask positioning and retaining device that secures the mask on the wear's face comprising:
a. a latching pad that is made from the hook component of a hook and loop fastener system,
b. a tape that adheres to the user's nose, and
c. a release pad located between the said adhesive tape and the user's skin that comprises a smaller profile than said latching pad, and operable to:
i. function as a spacer to elevate the outer edges of said latching pad above said adhesive tape,
ii. function as a chemical buffer between the latching pad/adhesive tape glue join,
iii. be mounted in a manner that provides a convenient grip on the apparatus to facilitate the removal of the device.

2. The device of claim 1, wherein said latching pad is comprised of material operable to engage with the inner surface of a mask.

3. The device of claim 1, wherein said latching pad is fastened to said mounting tape with a fastening system comprised of at least one of the following: stitches, rivets, laser fusion, ultrasonic welding, hot melt wax, rubber cement, epoxy, or adhesive glue.

4. The device of claim 1, wherein said latching pad further comprises a field of exposed latching hooks made of fine wire or cast plastic prongs protruding upwards

5. The device of claim 1, wherein said latching pad would be fastened to the mounting tape by at least one adhesive.

6. The device of claim 4, wherein said at least one adhesive is a pressure sensitive adhesive that is coated on the back side of the said latching pad.

7. The device of claim 1, wherein said mounting tape is comprised of a non-allergenic adhesive.

8. The device of claim 3, wherein said fastening system further comprises lamination or injection mold joining process.

9. The device of claim 1, wherein said latching pad further comprises a base comprised of said latching hooks and operable to provide a surface to latch onto said mounting tape.

10. The device of claim 9, wherein said latching pad is positioned above the mounting tape in such a manner that said latching pad overhangs said mounting tape creating a gap exposing the shape edges of said latching pad base increasing the gripping power of the device

11. The device of claim 1, wherein said release pad has exposed edges to provide a secure grip on the device to enable to wearer to safely remove the device.

12. A respirator face mask positioning and retaining device that secures the mask on the wear's face comprising:
a. a latching pad comprising a fastening system operable to affix the device to a face mask, and
b. a flexible band operable to adheres to the surface of a user's nose.

13. The device of claim 12, further comprising a padding layer located between said latching pad and said flexible band operable to elevate the latching pad and provide a grip for removing said device.

14. The device of claim 13, wherein said padding layer further comprises exposed edges operable to enable the user to safely and easily remove the device by providing a secure grip on the device.

15. The device of claim 12, further wherein said latching pad is coupled to said flexible band via at least one of the following affixation methods: stitches, rivets, laser fusion, ultrasonic welding, hot melt wax, rubber cement, epoxy, or adhesive glue.

16. The device of claim 12, wherein said fastening system comprises a field of exposed latching hooks that protrude outward from the superior side of said latching pad and are operable to latch onto the inner surface of a face mask.

17. The device of claim 12, wherein said affixation method comprises a pressure sensitive adhesive that is coated on the inferior side of the said latching pad.

18. The device of claim 12, wherein the inferior surface of said flexible band comprises a non-allergenic adhesive operable to adhere said flexible band to the surface of user's nose.

19. The device of claim 12, wherein said latching pad further comprises an exterior surface having latching hooks operable to latch onto the superior surface of said flexible band.
